(19) United States
(12) Patent Application Publication
(10) Pub. No.: US 2023/0061026 A1
(43) Pub. Date: Mar. 2, 2023

(54) TRAINING DATA GENERATION DEVICE, TRAINING DATA GENERATION METHOD, AND PROGRAMRECORDING MEDIUM

(71) Applicant: NEC Corporation, Minato-ku, Tokyo (JP)

(72) Inventors: Hiromi Shimizu, Tokyo (JP); Shinnosuke Nishimoto, Tokyo (JP); Junko Watanabe, Tokyo (JP); Riki Eto, Tokyo (JP); Noriyuki Tonouchi, Tokyo (JP); So Yamada, Tokyo (JP)

(73) Assignee: NEC Corporation, Minato-ku, Tokyo (JP)

(21) Appl. No.: 17/800,305

(22) PCT Filed: Mar. 13, 2020
(86) PCT No.: PCT/JP2020/010982

§ 371 Date: Aug. 17, 2022

(51) Int. Cl. G06N 20/00; G06V 10/774; G06F 40/279; G10L 15/06; G10L 15/26

(52) U.S. Cl. CPC G06N 20/00; G06V 10/774; G06F 40/279; G10L 15/063; G10L 15/26

(57) ABSTRACT

A training data generation device includes a label candidate generation unit, a reception unit, and a training data generation uni. The acquisition unit is configured to acquire smell data and information pertaining to the smell data. The label candidate generation unit which generates label candidates on the basis of the information pertaining to the smell data; an output unit which outputs the generated label candidates. The reception unit is configured to receive selection of a label from the output label candidates. The training data generation unit which generates training data from the selected label and the smell data.

### TECHNICAL FIELD

The present invention relates to a training data generation device, a training data generation method, a learning model generation method, and a program recording medium.

### BACKGROUND ART

PTL 1 discloses a technology for acquiring evaluation data by associating a detected indoor smell with a sensory evaluation of each user for the indoor smell.

### CITATION LIST

### Patent Literature

[PTL 1] WO 2018/168672 A

### SUMMARY OF INVENTION

### Technical Problem

In PTL 1, sensory evaluation choices prepared in advance are used as correct answer labels. Therefore, in the technology described in PTL 1, it is not possible to perform machine learning using a correct answer label other than the sensory evaluation choices prepared in advance.

An object of the present invention is to generate training data for performing machine learning using a desired correct answer label.

### Solution to Problem

A training data generation device of the present invention includes: acquisition means configured to acquire smell data and information regarding the smell data; label candidate generation means configured to generate label candidates based on the information regarding the smell data; output means configured to output the generated label candidates; reception means configured to receive selection of a label from the output label candidates; and training data generation means configured to generate training data based on the selected label and the smell data.

A training data generation method of the present invention includes: acquiring smell data and information regarding the smell data; generating label candidates based on the information regarding the smell data; outputting the generated label candidates; receiving selection of a label from the output label candidates; and generating training data based on the selected label and the smell data.

A learning model generation method of the present invention includes: acquiring smell data and information regarding the smell data; generating label candidates based on the information regarding the smell data; outputting the generated label candidates; receiving selection of a label from the output label candidates; generating training data based on the selected label and the smell data; and generating a learning model based on the generated training data.

A training data generation program recording medium of the present invention is a program recording medium that records a program for causing a computer to perform: processing of acquiring smell data and information regarding the smell data; processing of generating label candidates based on the information regarding the smell data; processing of outputting the generated label candidates; processing of receiving selection of a label from the output label candidates; and processing of generating training data based on the selected label and the smell data.

### Advantageous Effects of Invention

The present invention has an effect of generating training data for performing machine learning using a desired correct answer label.

### BRIEF DESCRIPTION OF DRAWINGS

FIG. 1 is a diagram illustrating a sensor 10 that detects a smell and time-series data obtained by the sensor 10 detecting a smell.

FIG. 2 is a schematic diagram of a prediction model.

FIG. 3 is a diagram schematically illustrating a training data generation system 100.

FIG. 4 is a diagram illustrating a functional configuration of a training data generation device 2000 according to a first example embodiment.

FIG. 5 is a diagram illustrating a computer for implementing the training data generation device 2000.

FIG. 6 is a diagram illustrating a flow of processing performed by the training data generation device 2000 according to the first example embodiment.

FIG. 7 is a view illustrating a screen for acquiring a speech as information regarding smell data, displayed on a terminal device 11.

FIG. 8 is a view illustrating a screen for selecting a label candidate, displayed on the terminal device 11.

FIG. 9 is a view illustrating a screen for acquiring an image as the information regarding the smell data, displayed on the terminal device 11.

FIG. 10 is a view illustrating a screen for receiving selection of a partial region including a measurement target, displayed on the terminal device 11.

FIG. 11 is a view illustrating a screen for receiving selection of a label, displayed on the terminal device 11.

FIG. 12 is a view illustrating a screen for acquiring a text as the information regarding the smell data, displayed on the terminal device 11.

FIG. 13 is a diagram illustrating training data stored in a storage unit 2010.

FIG. 14 is a diagram illustrating a functional configuration of a training data generation device 2000 according to a second example embodiment.

FIG. 15 is a diagram illustrating a flow of processing performed by the training data generation device 2000 according to the second example embodiment.

FIG. 16 is a diagram illustrating an outline of a trained model.

FIG. 17 is a diagram illustrating an example of a label space.

FIG. 18 is a diagram illustrating an outline of processing performed by a label candidate generation unit 2070.

FIG. 19 is a diagram illustrating a functional configuration of a training data generation device 2000 according to a third example embodiment.

FIG. 20 is a diagram illustrating a flow of processing performed by the training data generation device 2000 according to the third example embodiment.

FIG. 21 is a diagram illustrating a functional configuration of a training data generation device 2000 according to a fourth example embodiment.

FIG. 22 is a diagram illustrating a functional configuration of a training data generation device 2000 according to a fifth example embodiment.

### EXAMPLE EMBODIMENT

### First Example Embodiment

Hereinafter, a first example embodiment according to the present invention will be described.

<Sensor>

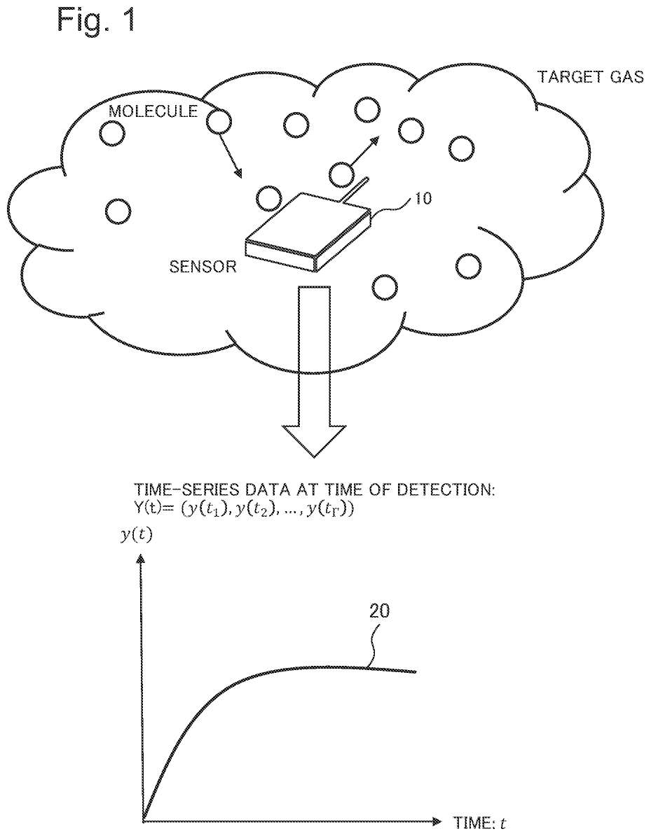

A sensor used in the present example embodiment will be described. FIG. 1 is a diagram illustrating a sensor 10 that detects a smell and time-series data obtained by the sensor 10 detecting a smell. The sensor 10 is a sensor including a receptor to which a molecule is to be attached, and a detection value changes according to attachment and detachment of the molecule to and from the receptor. A gas sensed by the sensor 10 is referred to as target gas. The time-series data of the detection value output from the sensor 10 is referred to as time-series data 20. Here, if necessary, the time-series data 20 is also referred to as Y, and the detection value at time t is also referred to as y(t). Y is a vector in which y(t) is listed.

For example, the sensor 10 may be a membrane-type surface stress sensor (MSS). The MSS includes, as the receptor, a functional film to which a molecule is to be attached, and stress generated in a support member of the functional film changes by attachment and detachment of the molecule to and from the functional film. The MSS outputs the detection value based on this change in stress. The sensor 10 is not limited to the MSS, and may be any sensor as long as it outputs the detection value based on a change in physical quantity related to viscoelasticity or a dynamic characteristic (mass, inertia moment, or the like) of a member of the sensor 10, which occurs according to attachment and detachment of a molecule to and from the receptor, and various types of sensors such as a cantilever type sensor, a membrane type sensor, an optical type sensor, a piezoelectric sensor, and a vibration response sensor can be adopted.

<Prediction Model>

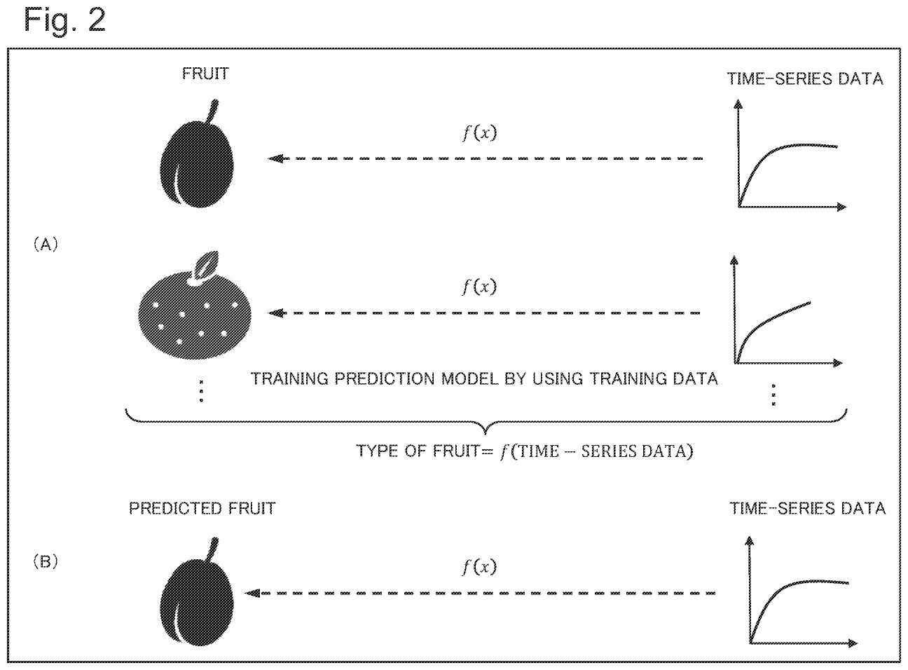

A prediction model used in the present example embodiment will be described. FIG. 2 is a schematic diagram of the prediction model. Here, a prediction model for predicting a fruit type based on the time-series data of the detection value output from the sensor 10 is illustrated as an example. FIG. 2(A) illustrates a phase in which the prediction model is trained. In FIG. 2(A), the prediction model is trained using, as training data, a combination of a certain fruit type (apple or the like) and the time-series data 20 of the detection value output from the sensor 10. FIG. 2(B) illustrates a phase in which the prediction model is used. In FIG. 2(B), the prediction model receives, as an input, time-series data acquired from a fruit of which type is unknown, and outputs the type of the fruit as a prediction result.

In the example embodiment described below, the prediction model is not limited to one that predicts a fruit type. The prediction model is only required to output a prediction result based on the time-series data of the detection value output from the sensor 10. For example, the prediction model may predict whether a person has contacted a specific disease based on exhalation of the person, may predict the presence or absence of a harmful substance from a smell in a house, or may predict an abnormality of factory equipment from a smell in a factory.

### Outline of Present Example Embodiment

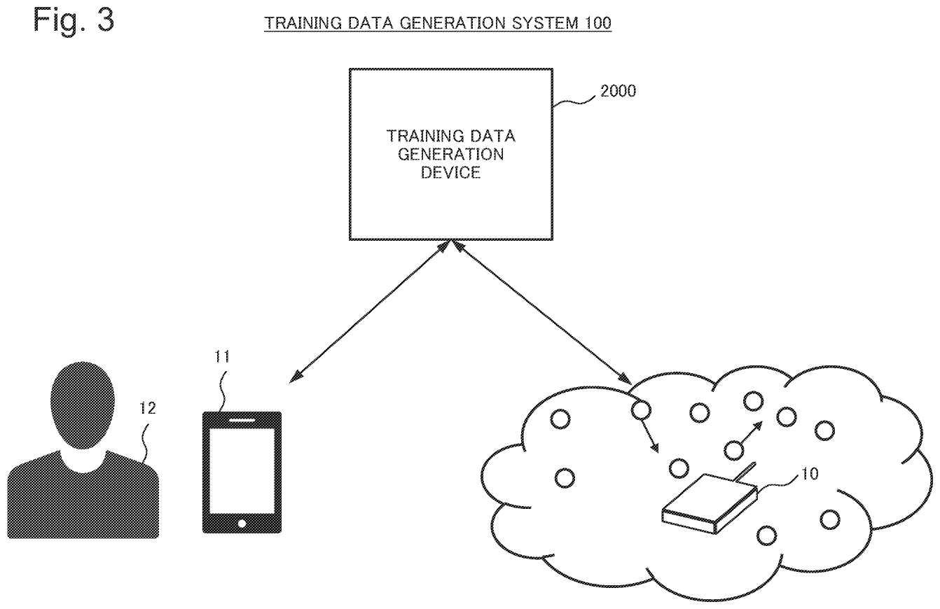

FIG. 3 is a diagram illustrating an outline of a training data generation system 100. The training data generation system 100 mainly includes a training data generation device 2000, the sensor 10 that acquires the time-series data by detecting a smell, and a terminal device 11 that receives information regarding the detected smell. The training data generation device 2000 and the sensor 10, and the training data generation device 2000 and the terminal device 11 perform data communication with each other via a communication network or the like. In FIG. 1, there is one sensor 10 and one terminal device 11, but there may be a plurality of sensors 10 and a plurality of terminal devices 11.

The training data generation device 2000 performs processing related to training data generation. Specifically, the training data generation device 2000 receives the time-series data (also referred to as “smell data”) from the sensor 10 and receives information regarding the smell data from an evaluator 12 through the terminal device 11. Details of the information regarding the smell data will be described later.

Here, the evaluator 12 refers to a person who inputs the information regarding the smell data and selects a label candidate to be described later. Hereinafter, in the present example embodiment, it is assumed that an evaluator who inputs the information regarding the smell data and an evaluator who selects the label candidate are the same person. However, the evaluator who inputs the information regarding the smell data and the evaluator who selects the label candidate may be different persons.

The training data generation device 2000 generates the label candidates to be assigned to the smell data based on the information regarding the smell data and outputs the label candidates to the terminal device 11. The terminal device 11 displays the label candidates on the screen and receives selection of a label from the evaluator 12. The terminal device 11 outputs the received label to the training data generation device 2000. The training data generation device 2000 generates the training data by combining the received label and the smell data.

<Example of Functional Configuration of Training Data Generation Device 2000>

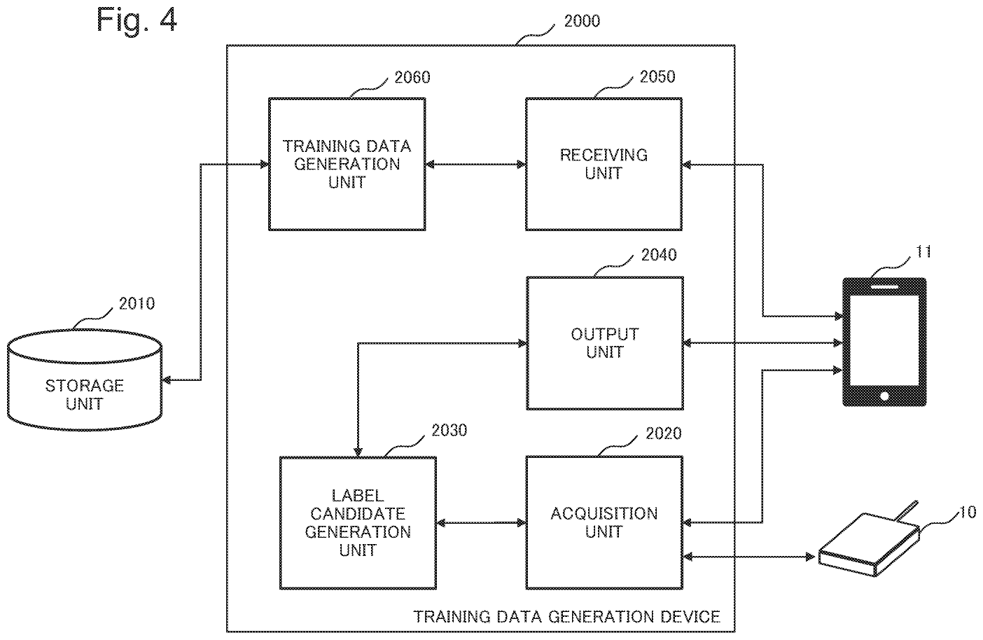

FIG. 4 is a diagram illustrating a functional configuration of the training data generation device 2000 according to the first example embodiment. The training data generation device 2000 includes an acquisition unit 2020, a label candidate generation unit 2030, an output unit 2040, a receiving unit 2050, and a training data generation unit 2060. The acquisition unit 2020 acquires the smell data from the sensor 10 and acquires the information regarding the smell data from the terminal device 11. The label candidate generation unit 2030 generates the label candidates based on the information regarding the smell data. The output unit 2040 outputs the label candidates generated by the label candidate generation unit 2030 to the terminal device 11. The receiving unit 2050 receives selection of a label from the terminal device 11. The training data generation unit 2060 generates the training data based on the selected label and the smell data, and outputs the training data to the storage unit 2010.

<Hardware Configuration of Training Data Generation Device 2000>

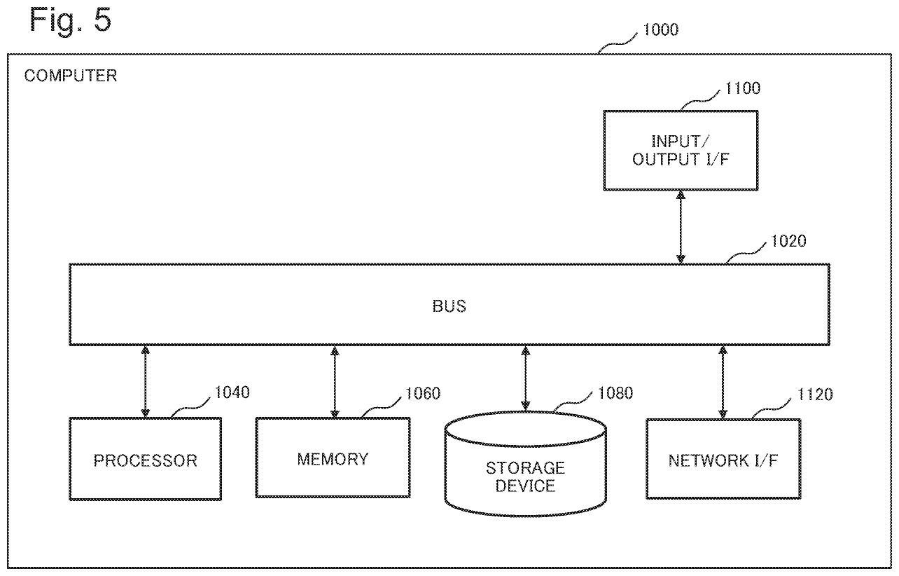

FIG. 5 is a diagram illustrating a computer for implementing the training data generation device 2000 illustrated in FIGS. 3 and 4. A computer 1000 is an arbitrary computer. For example, the computer 1000 is a stationary computer such as a personal computer (PC) or a server machine. In addition, for example, the computer 1000 is a portable computer such as a smartphone or a tablet terminal. The computer 1000 may be a dedicated computer designed to implement the training data generation device 2000 or may be a general-purpose computer.

The computer 1000 includes a bus 1020, a processor 1040, a memory 1060, a storage device 1080, an input/output interface 1100, and a network interface 1120. The bus 1020 is a data transmission path for the processor 1040, the memory 1060, the storage device 1080, the input/output interface 1100, and the network interface 1120 to transmit and receive data to and from each other. However, a method of connecting the processor 1040 and the like to each other is not limited to the bus connection.

The processor 1040 is various processors such as a central processing unit (CPU), a graphics processing unit (GPU), and a field-programmable gate array (FPGA). The memory 1060 is a main storage device implemented by using a random access memory (RAM) or the like. The storage device 1080 is an auxiliary storage device implemented by using a hard disk, a solid state drive (SSD), a memory card, a read only memory (ROM), or the like.

The input/output interface 1100 is an interface for connecting the computer 1000 and input/output devices. For example, an input device such as a keyboard and an output device such as a display device are connected to the input/output interface 1100. In addition, for example, the sensor 10 is connected to the input/output interface 1100. However, the sensor 10 is not necessarily directly connected to the computer 1000. For example, the sensor 10 may store acquired data in a storage device shared with the computer 1000.

The network interface 1120 is an interface for connecting the computer 1000 to a communication network. The communication network is, for example, a local area network (LAN) or a wide area network (WAN). A method of connecting the network interface 1120 to the communication network may be wireless connection or wired connection.

The storage device 1080 stores program modules that implement the functional configuration units of the training data generation device 2000. The processor 1040 reads the program modules to the memory 1060 and executes the program modules, thereby implementing the functions relevant to the program modules.

<Flow of Processing>

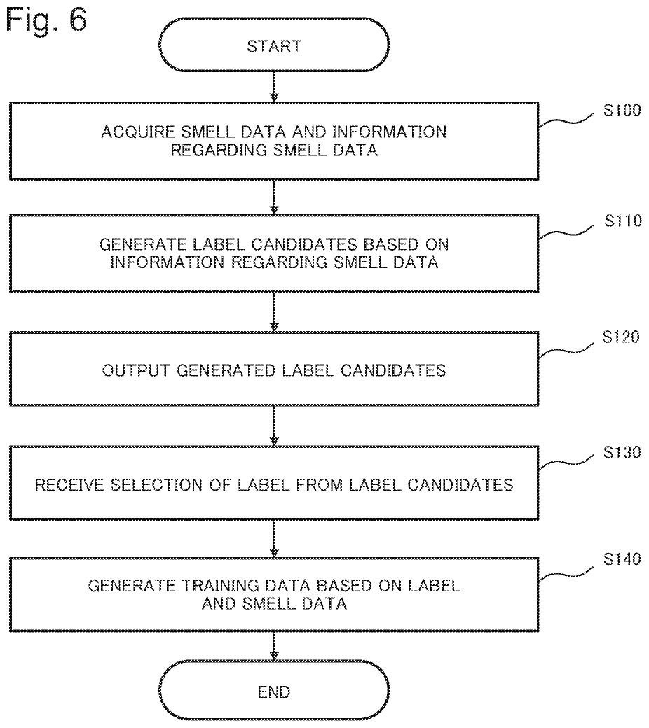

FIG. 6 is a diagram illustrating a flow of the processing performed by the training data generation device 2000 according to the first example embodiment. The acquisition unit 2020 acquires the smell data and the information regarding the smell data (S100). The label candidate generation unit 2030 generates the label candidates based on the information regarding the smell data (S110). The output unit 2040 outputs the generated label candidates to the terminal device 11 (S120). The receiving unit 2050 receives selection of a label from the label candidates (S130). The training data generation unit 2060 generates the training data based on the selected label and the smell data (S140).

<Case Where Information Regarding Smell Data is Speech>

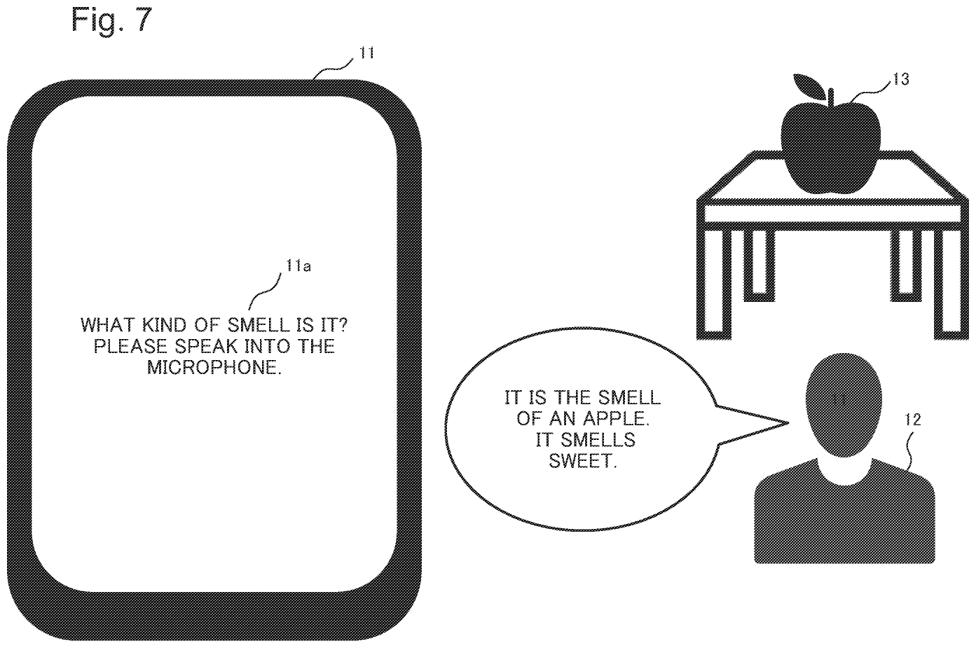

The operation of the training data generation device 2000 in a case where the information regarding the smell data is a speech will be described with reference to FIGS. 7, 8, and 9. FIG. 7 is a view illustrating a screen for acquiring a speech as the information regarding the smell data, displayed on the terminal device 11. The screen illustrated in FIG. 7 includes, for example, a message 11a (for example, “What kind of smell is it? Please speak into the microphone.”) requesting smell evaluation.

The evaluator 12 inputs a speech indicating an evaluation of the smell of a measurement target 13 (for example, “It is the smell of an apple. It smells sweet.”) to the terminal device 11. The terminal device 11 outputs the received speech to the acquisition unit 2020. The acquisition unit 2020 outputs the acquired speech to the label candidate generation unit 2030.

Processing in which the label candidate generation unit 2030 generates the label candidates based on the acquired speech will be described. The label candidate generation unit 2030 converts the acquired speech into a text by using an existing speech recognition technology. The label candidate generation unit 2030 generates the label candidates by applying an existing natural language processing technology to the converted text. Examples of the natural language processing technology for generating the label candidates include a method using character string matching based on an expression dictionary, term frequency-inverse document frequency (TF-IDF), Key-Graph, and a known machine learning technology. The label candidate generation unit 2030 outputs the text obtained by the conversion and the generated label candidates to the output unit 2040. The output unit 2040 outputs the text obtained by the conversion and the generated label candidates to the terminal device 11.

Here, an example of a method in which the label candidate generation unit 2030 generates the label candidates by using the natural language processing technology will be described. First, the label candidate generation unit 2030 performs morphological analysis on the text converted from the speech and acquires work class information of words included in the text. Next, the label candidate generation unit 2030 acquires, as the label candidate, a word to which a predetermined word class (“noun”, “adjective”, or the like) is given among the words included in the text.

A method of determining the predetermined word class used by the label candidate generation unit 2030 is not limited. For example, the label candidate generation unit 2030 may further receive a task setting of machine learning from the evaluator 12 and determine a predetermined word class based on the received task setting. Specifically, in a case where the task setting received from the evaluator 12 is “object identification”, the label candidate generation unit 2030 acquires, as the label candidate, a word to which a word class (“noun”, “proper noun”, or the like) that can represent the name of the object is assigned. In a case where the task setting received from the evaluator 12 is “polarity classification”, the label candidate generation unit 2030 acquires, as the label candidate, a word to which a word class (“adjective”, “adverb”, or the like) that can affect the polarity of the text is assigned.

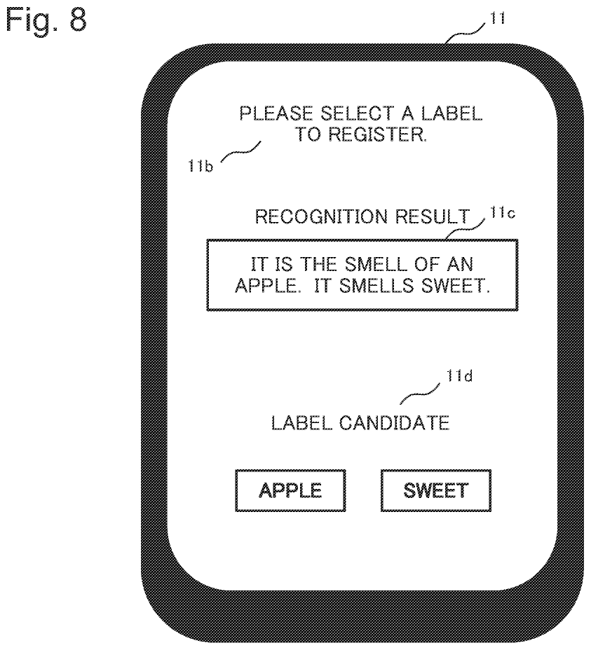

FIG. 8 is a view illustrating a screen for selecting a label candidate, displayed on the terminal device 11. The screen illustrated in FIG. 8 includes, for example, a message 11b (for example, “Please select a label to register.”) suggesting selection of a label to register, a speech recognition result 11c, and label candidates 11d. The speech recognition result 11c is a speech recognition result related to the smell evaluation input by the evaluator 12. The label candidates 11d are buttons indicating the label candidates (for example, “apple” and “sweet”) generated by the label candidate generation unit 2030.

For example, the evaluator 12 selects a label by pressing a button of a label to register from the label candidates 11d. The acquisition unit 2020 acquires the selected label.

The label candidate 11d illustrated in FIG. 8 may include “none”. In a case where there is no appropriate label among the label candidates 11d, the evaluator 12 selects “none”. In this case, for example, the operation illustrated in FIG. 7 is performed again.

<Case Where Information Regarding Smell Data is Image>

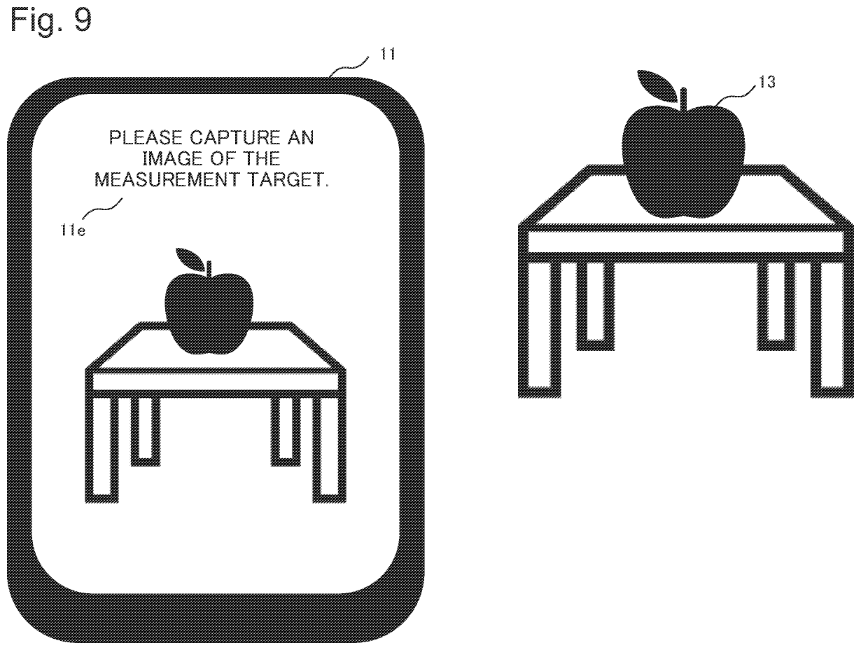

An operation of the training data generation device 2000 in a case where the information regarding the smell data is an image will be described with reference to FIGS. 9, 10, and 11. FIG. 9 is a view illustrating a screen for acquiring an image as the information regarding the smell data, displayed on the terminal device 11. The screen illustrated in FIG. 9 includes, for example, a message 11e (for example, “Please capture an image of the measurement target.”) instructing imaging of the measurement target 13.

The evaluator 12 images the measurement target 13 by using an imaging device provided in the terminal device 11. The terminal device 11 outputs the captured image to the acquisition unit 2020. The acquisition unit 2020 outputs the acquired speech to the label candidate generation unit 2030.

Processing in which the label candidate generation unit 2030 generates the label candidates based on the acquired image will be described. The label candidate generation unit 2030 extracts, from the acquired image, a partial region that is a region candidate including the measurement target by using an existing image recognition technology. Examples of the image recognition technology for extracting the partial region include a sliding window method, a binarized normed gradients (BING), a selective search method, a branch and bound method, and the like. The label candidate generation unit 2030 outputs the extracted partial region to the output unit 2040. The output unit 2040 outputs the extracted partial region to the terminal device 11.

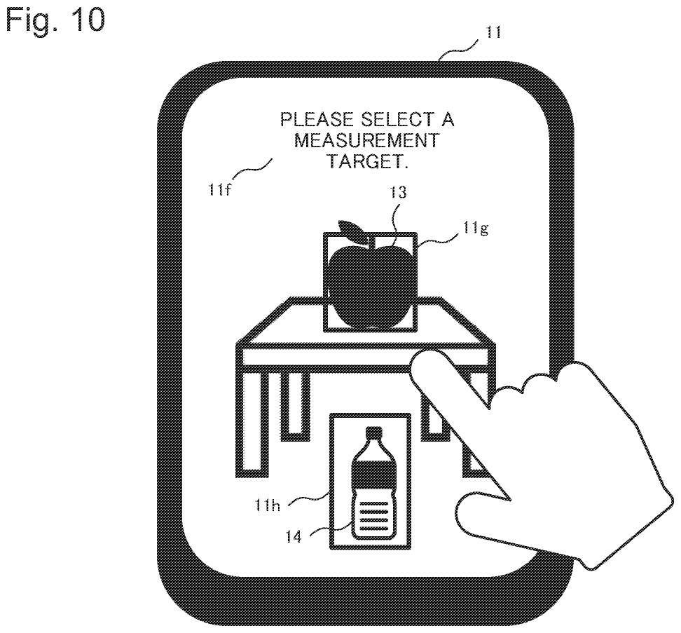

FIG. 10 is a view illustrating a screen for receiving selection of the partial region including the measurement target, displayed on the terminal device 11. The screen illustrated in FIG. 10 includes, for example, a message 11f (for example, “Please select a measurement target.”) suggesting selection of the partial region including the measurement target, an extracted partial region 11g, and an extracted partial region 11h.

The evaluator 12 selects the partial region including the measurement target 13 among the displayed partial regions. The terminal device 11 outputs the selected partial region to the receiving unit 2050.

The label candidate generation unit 2030 generates the label candidates for the acquired partial region by using an existing image recognition technology. Examples of the image recognition technology for generating the label candidates include methods using a linear classifier, ensemble learning, and a nonlinear classifier such as a convolutional neural network. The label candidate generation unit 2030 outputs the generated label candidates to the output unit 2040. The output unit 2040 outputs the label candidates to the terminal device 11.

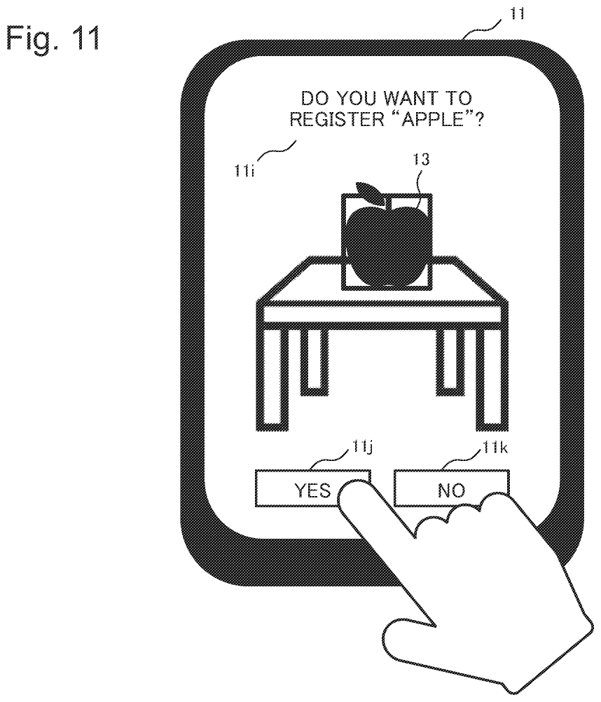

FIG. 11 is a view illustrating a screen for receiving selection of a label, displayed on the terminal device 11. The screen illustrated in FIG. 11 includes, for example, a message 11i (for example, “Do you want to register “apple”?”) indicating selection of a label, a selection button “Yes” 11j, and a selection button “No” 11k.

The evaluator 12 presses the selection button “Yes” 11j to select the displayed label candidate, and presses the selection button “No” 11k to select no label candidate. In a case where the evaluator 12 has pressed the selection button “Yes” 11j, the terminal device 11 outputs the selected label to the receiving unit 2050. In a case where the evaluator 12 has pressed the selection button “No” 11k, the terminal device 11 may display the instruction to image the measurement target 13 illustrated in FIG. 9 on the screen again.

In the screen illustrated in FIG. 11, selection of whether one label candidate is selectable is received. However, in a case where the label candidate generation unit 2030 generates a plurality of label candidates based on an image, the screen illustrated in FIG. 11 may display the plurality of label candidates. In this case, the evaluator 12 selects one or more labels from the displayed label candidates. Then, the terminal device 11 outputs the selected labels to the receiving unit 2050.

<Case Where Information Regarding Smell Data is Text>

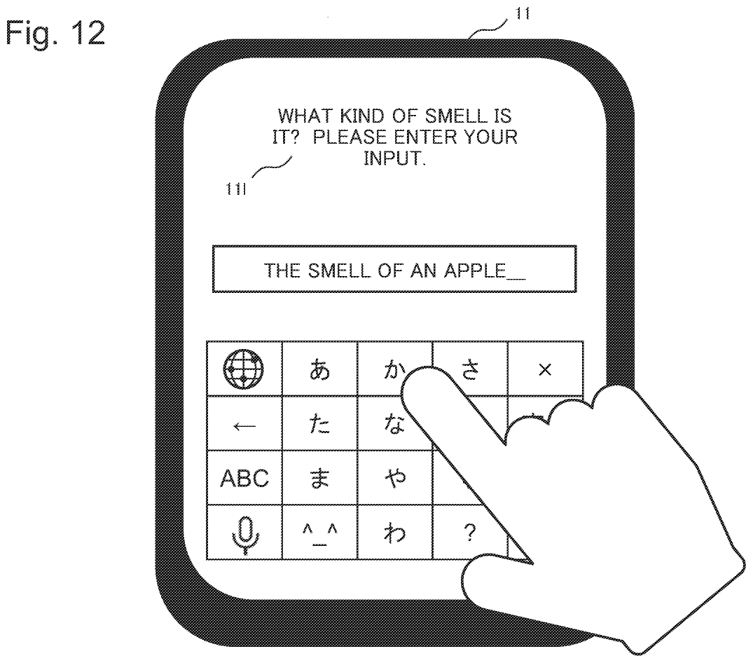

An operation of the training data generation device 2000 in a case where the information regarding the smell data is a text will be described with reference to FIG. 12. FIG. 12 is a view illustrating a screen for acquiring a text as the information regarding the smell data, displayed on the terminal device 11. The screen illustrated in FIG. 12 includes, for example, a message 111 (for example, “What kind of smell is it? Please enter your input.”) requesting an evaluation of the smell of the measurement target 13.

The evaluator 12 inputs the evaluation of the smell of the measurement target 13 (for example, “The smell of an apple”) by using a keyboard displayed on the screen. The terminal device 11 outputs the received text to the acquisition unit 2020. The acquisition unit 2020 outputs the acquired sentence to the label candidate generation unit 2030.

Processing in which the label candidate generation unit 2030 generates the label candidates based on the acquired sentence is similar to the processing after a speech is converted into a text in a case where the information regarding the smell data is a speech.

<Generated Training Data>

Processing in which the training data generation unit 2060 generates the training data will be described. The training data generation unit 2060 generates the training data by associating the selected label with the smell data, and outputs the training data to the storage unit 2010.

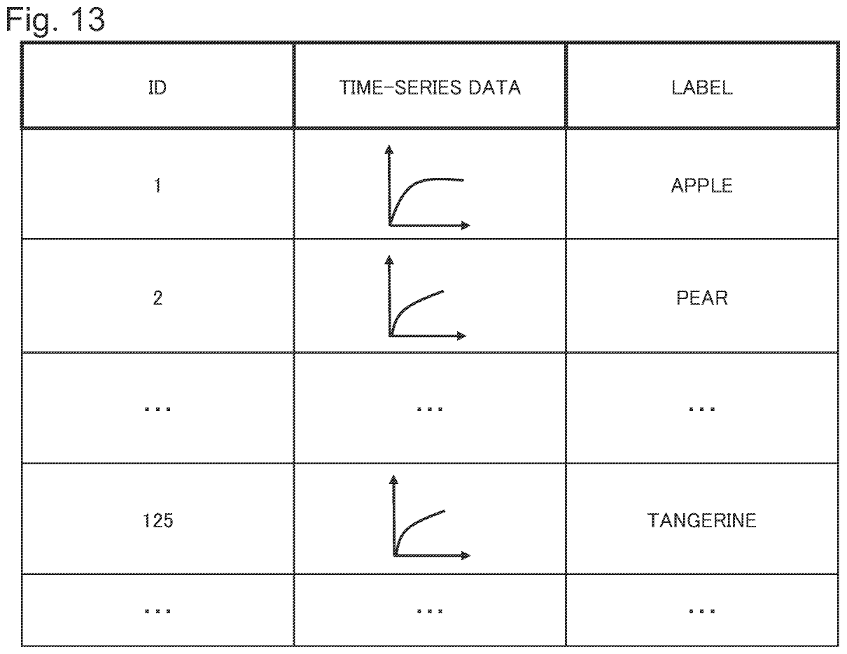

FIG. 13 is a diagram illustrating the training data stored in the storage unit 2010. Each record in FIG. 13 is relevant to the training data. Each piece of training data includes, for example, an ID for identifying the training data, the smell data obtained by the sensor 10 detecting the smell, and the selected label.

Each record may include a sensor ID for identifying the sensor 10 that has detected the smell, a measurement date, the measurement target, and a measurement environment.

The measurement date may be, for example, a date on which the target gas is injected into the sensor 10 or a date on which the generated training data is stored in the storage unit 2010. The measurement date may be a measurement date and time including a measurement time.

The measurement environment is information regarding an environment at the time of measuring the smell. For example, the measurement environment includes the temperature, humidity, and sampling interval of the environment in which the sensor 10 is installed.

The sampling interval indicates an interval at which the smell is measured, and is expressed as Δt [s] or a sampling frequency [Hz] using a reciprocal of Δt [s]. For example, the sampling interval is 0.1 [s], 0.01 [s], or the like.

In a case where the smell is measured by alternately injecting sample gas and purge gas to the sensor 10, the sample gas and the purge gas injection time may be set as the sampling interval. Here, the sample gas is the target gas in FIG. 1. The purge gas is gas (for example, nitrogen) for removing the target gas attached to the sensor 10. For example, the sensor 10 can measure data by injecting the sample gas for five seconds and the purge gas for five seconds.

The measurement environment such as the temperature, humidity, and sampling interval described above may be acquired by, for example, a meter provided inside or outside the sensor 10, or may be input from a user through the terminal device 11.

In the present example embodiment, the temperature, the humidity, and the sampling interval have been described as examples of the measurement environment, but examples of other measurement environments include information on a distance between the measurement target and the sensor 10, the type of purge gas, carrier gas, the type of the sensor (the sensor ID and the like), the season at the time of measurement, the atmospheric pressure at the time of measurement, the atmosphere (CO₂ concentration and the like) at the time of measurement, and a measurer. The carrier gas is gas injected simultaneously with the smell to be measured, and for example, nitrogen or the atmosphere is used. The sample gas is a mixture of the carrier gas and the smell to be measured.

The above-described temperature and humidity may be acquired from a setting value of the measurement target, the carrier gas, the purge gas, the sensor 10 itself, the atmosphere around the sensor 10, the sensor 10, or a device that controls the sensor 10.

<Actions and Effects>

The training data generation device 2000 according to the present example embodiment has an effect of generating the label candidates based on the information regarding the smell data and generating the training data for performing machine learning using a desired correct answer label by associating the label selected by the evaluator 12 with the smell data.

### Second Example Embodiment

Hereinafter, a second example embodiment according to the present invention will be described. The second example embodiment is different from the first example embodiment in that a label candidate generation unit 2070 generates label candidates based on a trained model. Details will be described below.

<Example of Functional Configuration of Training Data Generation Device 2000>

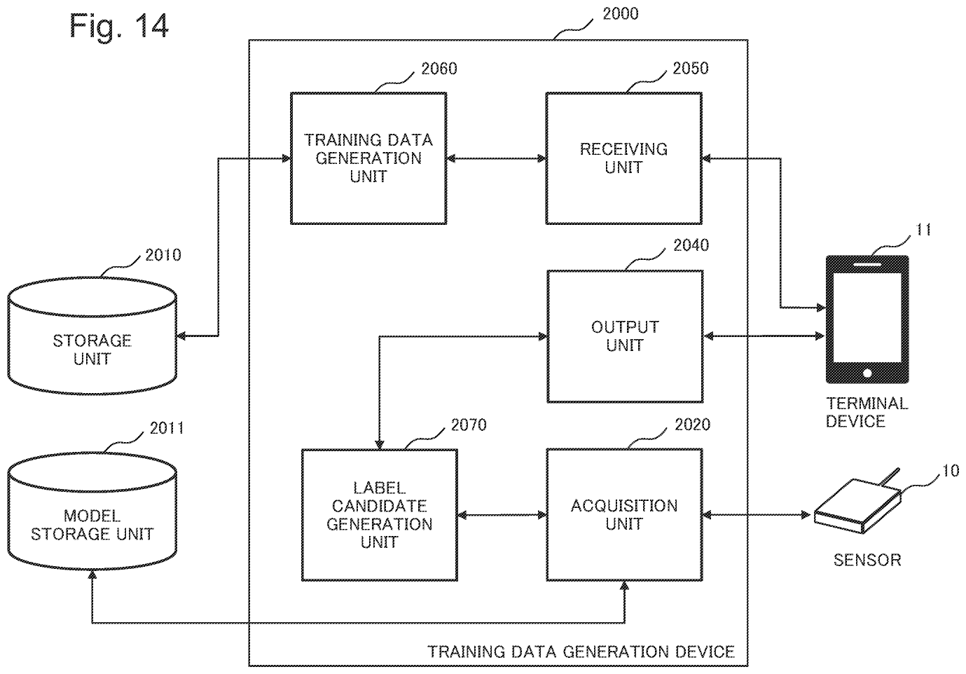

FIG. 14 is a diagram illustrating a functional configuration of a training data generation device 2000 according to the second example embodiment. The training data generation device 2000 according to the second example embodiment includes an acquisition unit 2020, the label candidate generation unit 2070, an output unit 2040, a receiving unit 2050, and a training data generation unit 2060. The acquisition unit 2020 acquires smell data from a sensor 10 and acquires a trained model to be described later from a model storage unit 2011. The label candidate generation unit 2070 generates the label candidates based on the acquired smell data and trained model. The operations of the output unit 2040, the receiving unit 2050, and the training data generation unit 2060 are similar to those in other example embodiments, and a description thereof will be omitted in the present example embodiment.

<Flow of Processing>

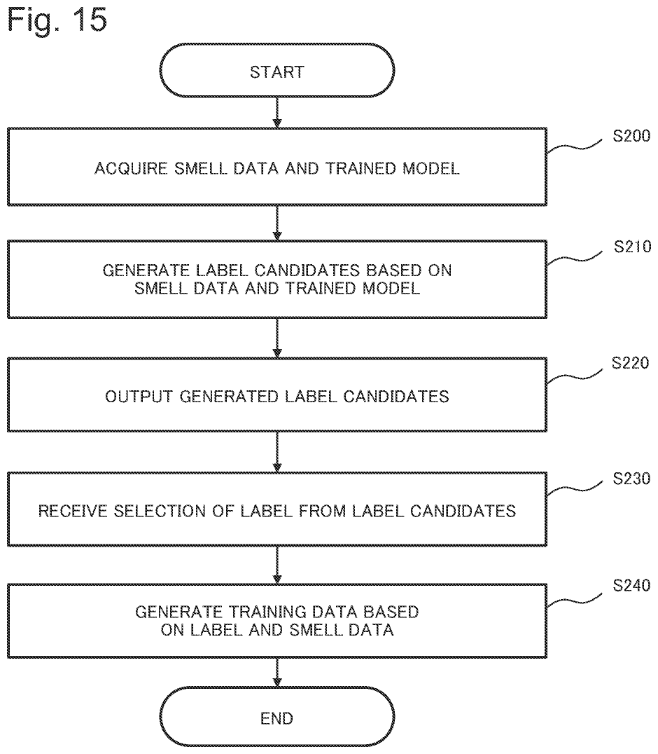

FIG. 15 is a diagram illustrating a flow of processing performed by the training data generation device 2000 according to the second example embodiment. The acquisition unit 2020 acquires the smell data and the trained model (S200). The label candidate generation unit 2070 generates the label candidates based on the smell data and the trained model (S210). The pieces of processing related to S220, S230, and S240 are similar to those in other example embodiments, and a description thereof will be omitted in the present example embodiment.

<Outline of Trained Model>

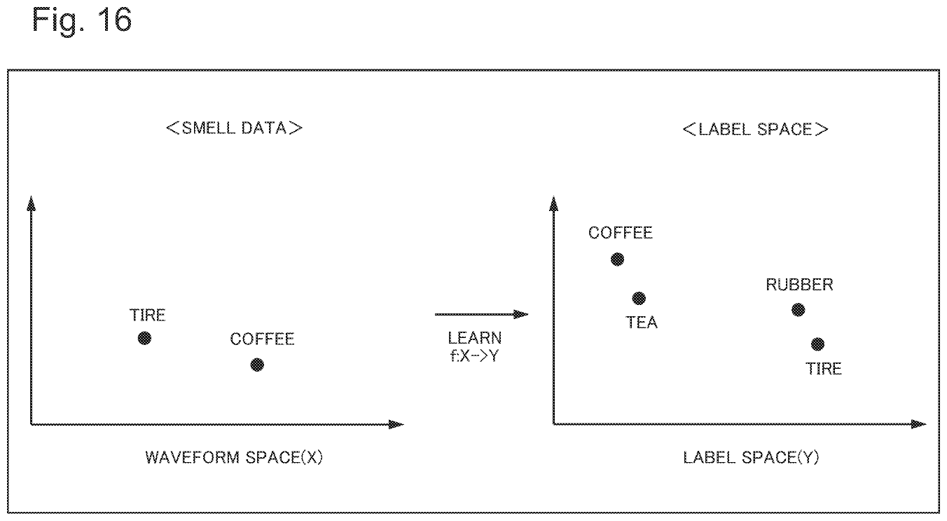

Details of the trained model stored in the model storage unit 2011 will be described. FIG. 16 is a diagram illustrating an outline of the trained model. The trained model is a machine learning model that assigns a value on a label space to a value on a waveform space that defines a feature amount of the smell data. Details of the label space will be described later.

As a training method for the trained model, there is a known machine learning method such as a deep learning model. For example, in a case where the trained model is a model trained by supervised machine learning, the training data is data in which a value indicating “coffee” in the waveform space illustrated in FIG. 16 is associated with a value indicating “coffee” in the label space.

A description of the label space is provided below. The label space is a vector space indicating the feature of the smell, and is a space in which a value obtained as a prediction result of the trained model is defined. It is possible to quantitatively express a relationship between a plurality of smells by expressing the smell by using the value of the label space. For example, labels located close to each other in a certain label space, such as “coffee” and “tea” or “rubber” and “tire” in the label space illustrated in FIG. 16, indicate similar smells in the label space. Labels located away from each other in a certain label space, such as “coffee” and “tire” or “tea” and “rubber” in the label space illustrated in FIG. 16, can be considered to indicate smells having contrasting properties in the label space. However, even the same smell is represented by different values in different label spaces. In the present example embodiment, as described below, a trained model using a plurality of label spaces can be used.

<Trained Model Using Space Indicating Structure or Chemical Property of Substance>

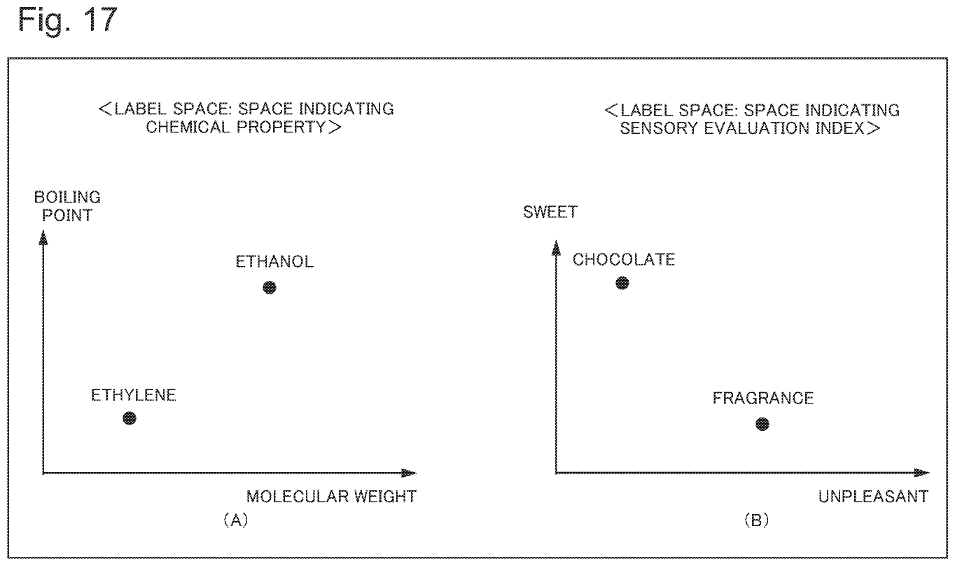

A case where the label space of the trained model is a space defined by a structure or chemical property of a substance will be described. FIG. 17 is a diagram illustrating the label space. FIG. 17(A) illustrates an example in which a vector space having a “molecular weight” and a “boiling point”, which are chemical properties of a substance, as axes is used as the label space, and labels “ethylene” and “ethanol” are expressed on this space. Examples of usable axes other than the molecular weight and the boiling point include a composition formula, a rational formula, a structural formula, the type and number of functional groups, the number of carbon atoms, the degree of unsaturation, a concentration, solubility in water, polarity, a melting point, a density, a molecular orbital, and the like. The space indicating a structure or chemical property of a substance may be a space defined by mol2vec which is a method of expressing a molecular structure by a high-dimensional real number vector.

<Trained Model Using Space Indicating Sensory Evaluation Index>

A case where the label space of the trained model is a space defined by an index (sensory evaluation index) obtained in an inspection for determining a target smell using human senses will be described. FIG. 17(B) illustrates an example in which a vector space having “unpleasant” and “sweet”, which are examples of the sensory evaluation index, as axes is used as the label space, and labels “chocolate” and “fragrance” are expressed on this space. Examples of the sensory evaluation include a discriminative test such as a two-point discrimination method or a three-point discrimination method, a descriptive test such as a scoring method or a quantitative descriptive analysis (QDA) method, a time intensity test, a time-dynamic method such as temporal dominance of sensations (TDS) or temporal check-all-that-apply (TCATA), and a palatable sensory evaluation method using a general panel.

<Trained Model Using Space Indicating Reaction When Sniffing Smell>

A case where the label space of the trained model is a space defined by a biological reaction that occurs in a human body when the human sniffs a smell is described. Examples of the biological reaction include electroencephalogram, a functional magnetic resonance imaging (fMRI) image, and an R-R Interval (RRI) when a human sniffs a smell. The label space is a waveform space that defines the feature amount of the biological reaction.

<Trained Model Using Word Embedding Space>

A case where the label space of the trained model is a space defined by word embedding (word distributed representation) will be described. The word embedding (word distributed representation) is a method of representing the meaning of a word as a high-dimensional real number vector, and methods such as word2vec, GloVe, fastText, and bidirectional encoder representations from transformers (BERT) are known.

However, since the nature of word embedding (word distributed representation) depends on a sentence (corpus) used when learning the word embedding, in a case where the word embedding space is used as the label space of the trained model, it is necessary to learn the word embedding (word distributed representation) using a sentence related to a smell. Examples of the sentence related to the smell include a research document such as a paper regarding olfaction, a cosmetic review, a food catalog, a gourmet article, and the like.

<Example of Operation of Label Candidate Generation Unit 2070>

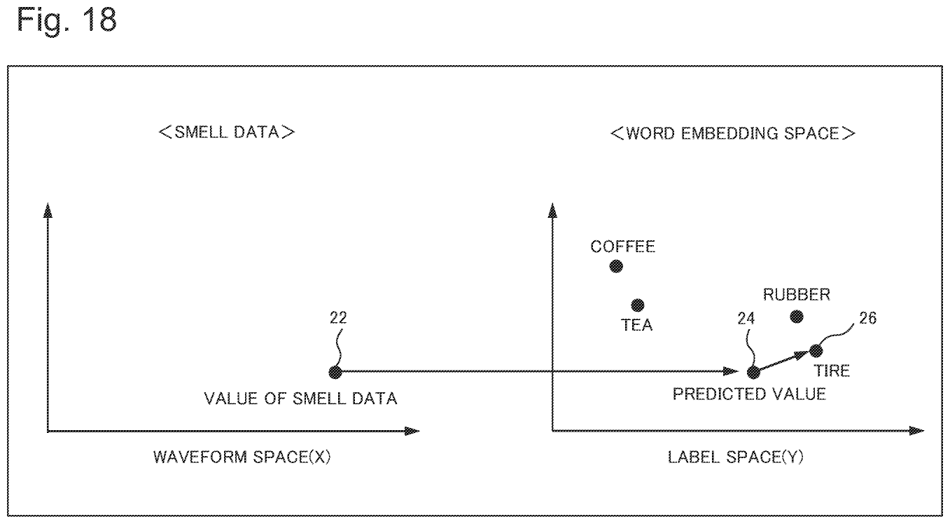

FIG. 18 is a diagram illustrating an outline of processing performed by the label candidate generation unit 2070. The processing performed by the label candidate generation unit 2070 will be specifically described with reference to FIG. 18. Here, a case where the label candidate generation unit 2070 uses the trained model using the word embedding space will be described as an example.

As illustrated in FIG. 18, the label candidate generation unit 2070 acquires the smell data from the acquisition unit 2020. The label candidate generation unit 2070 calculates the feature amount of the acquired smell data. As illustrated in FIG. 18, the calculated feature amount is relevant to a value 22 indicating the acquired smell data in the waveform space. Next, the label candidate generation unit 2070 calculates a predicted value 24 in the label space by using the value 22 indicating the smell data and the trained model. Then, the label candidate generation unit 2070 performs nearest neighbor search, and acquires, for example, a point 26 relevant to “tire” as a neighboring point of the predicted value. The label candidate generation unit 2070 generates “tire” as the label candidate.

Examples of a method of calculating the feature amount of the smell data by the label candidate generation unit 2070 include an average value of the smell data obtained by detecting the measurement target a plurality of times using the sensor 10, a value indicating a feature in the shape of the detection value, and a value, a maximum value, a minimum value, a median value, and the like of a component configuration when the smell data is decomposed into exponential components. The label candidate generation unit 2070 may use the value of the acquired smell data as the feature amount.

The number of label candidates acquired by the label candidate generation unit 2070 is not limited to one. For example, the label candidate generation unit 2070 may acquire a plurality of neighboring points using a K-nearest neighbors algorithm and generate a plurality of label candidates.

<Actions and Effects>

The training data generation device 2000 according to the present example embodiment generates label candidates using a trained model that associates smell data with a vector space indicating the feature of the smell. That is, since the training data generation device 2000 can generate the label candidates in quantitative consideration of a relationship between a plurality of smells, there is an effect of generating the training data for performing machine learning using a desired correct answer label.

### Third Example Embodiment

Hereinafter, a third example embodiment according to the present invention will be described. The third example embodiment is different from other example embodiments in that a learning unit 2080 is included. Details will be described below.

<Example of Functional Configuration of Training Data Generation Device 2000>

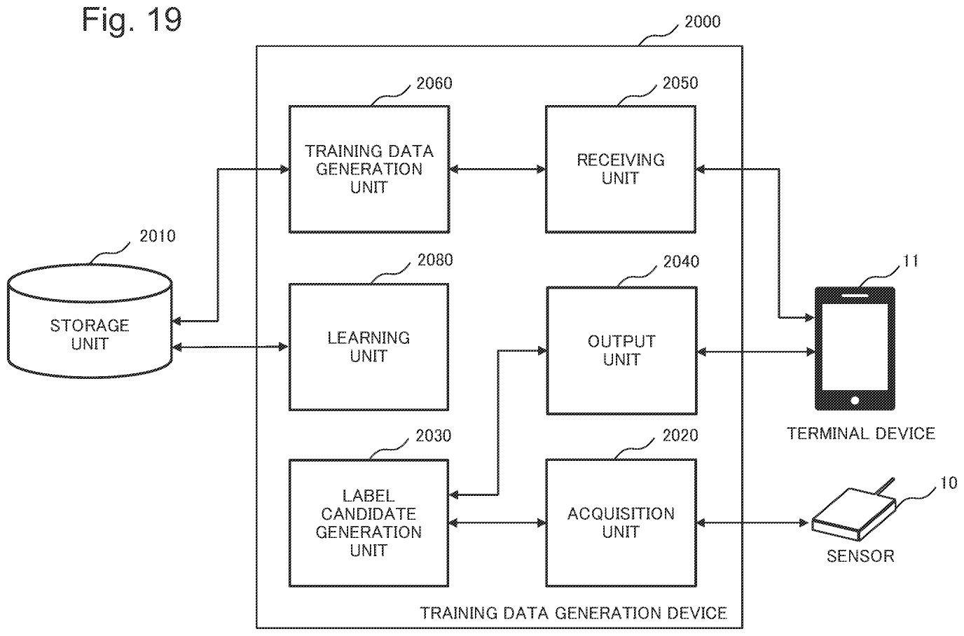

FIG. 19 is a diagram illustrating a functional configuration of a training data generation device 2000 according to the third example embodiment. The training data generation device 2000 according to the third example embodiment includes an acquisition unit 2020, a label candidate generation unit 2030, an output unit 2040, a receiving unit 2050, a training data generation unit 2060, and the learning unit 2080. The learning unit 2080 acquires the training data from a storage unit 2010 and performs machine learning. The operations of the acquisition unit 2020, the label candidate generation unit 2030, the output unit 2040, the receiving unit 2050, and the training data generation unit 2060 are similar to those in other example embodiments, and a description thereof will be omitted in the present example embodiment.

<Flow of Processing>

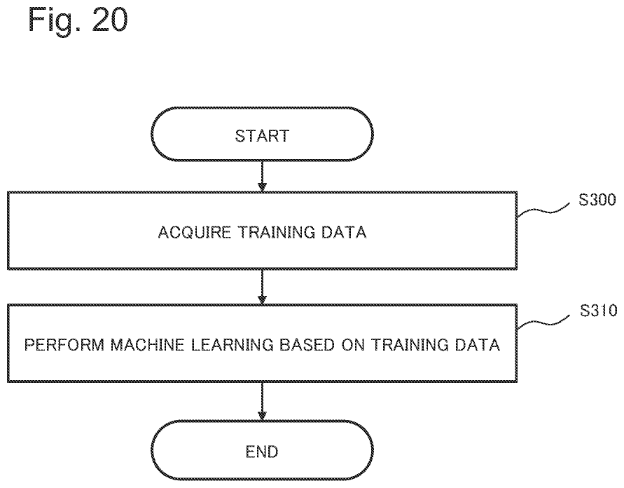

FIG. 20 is a diagram illustrating a flow of processing performed by the training data generation device 2000 according to the third example embodiment. The learning unit 2080 acquires the training data (S300). The learning unit 2080 performs machine learning based on the acquired training data (S310). A method in which the learning unit 2080 performs machine learning includes deep learning, a support vector machine (SVM), and the like, and is not particularly limited.

### Fourth Example Embodiment

Hereinafter, a fourth example embodiment according to the present invention will be described.

<Example of Functional Configuration of Training Data Generation Device 2000>

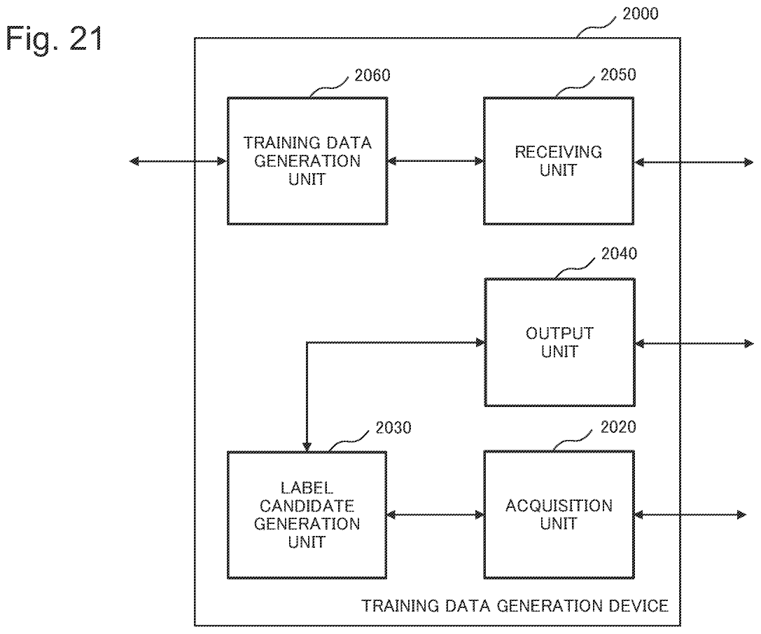

FIG. 21 is a diagram illustrating a functional configuration of a training data generation device 2000 according to the fourth example embodiment. The training data generation device 2000 according to the second example embodiment includes an acquisition unit 2020, a label candidate generation unit 2030, an output unit 2040, a receiving unit 2050, and a training data generation unit 2060. The operation of each unit is similar to that of other example embodiments, and a description thereof will be omitted in the present example embodiment.

### Fifth Example Embodiment

Hereinafter, a fifth example embodiment according to the present invention will be described.

<Example of Functional Configuration of Training Data Generation Device 2000>

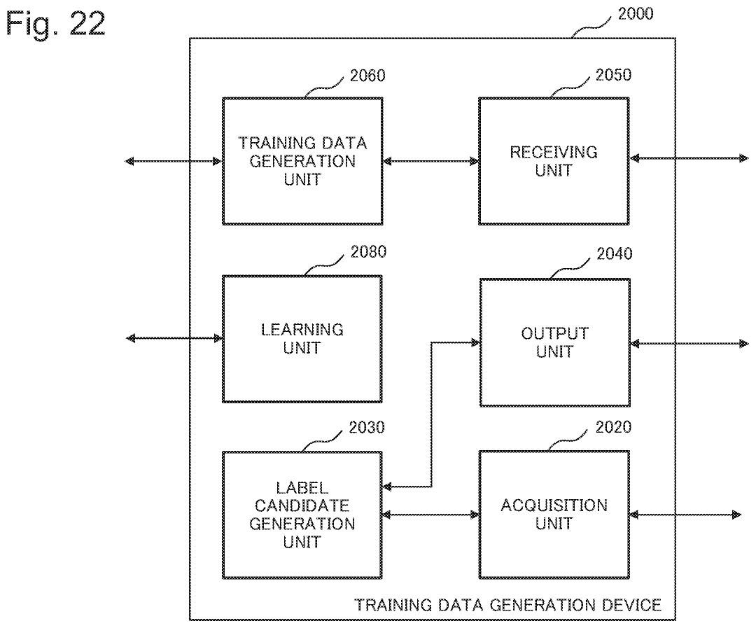

FIG. 22 is a diagram illustrating a functional configuration of a training data generation device 2000 according to the fifth example embodiment. The training data generation device 2000 according to the fifth example embodiment includes an acquisition unit 2020, a label candidate generation unit 2030, an output unit 2040, a receiving unit 2050, a training data generation unit 2060, and a learning unit 2080. The operation of each unit is similar to that of other example embodiments, and a description thereof will be omitted in the present example embodiment.

The present invention is not limited to the above-described example embodiments and can be embodied by modifying the constituent elements without departing from the gist thereof at the implementation stage. In addition, various inventions can be made by appropriately combining a plurality of constituent elements disclosed in the above-described example embodiments. For example, some constituent elements may be deleted from all the constituent elements of the example embodiments. Furthermore, the constituent elements of different example embodiments may be appropriately combined.

<Supplementary Note>

Some or all of the above-described example embodiments can also be described as the following Supplementary Notes. Hereinafter, an outline of a replication method and the like in the present invention will be described. However, the present invention is not limited to the following configuration.

(Supplementary Note 1)

A training data generation device including:

acquisition means configured to acquire smell data and information regarding the smell data;

label candidate generation means configured to generate label candidates based on the information regarding the smell data;

output means configured to output the generated label candidates;

reception means configured to receive selection of a label from the output label candidates; and

training data generation means configured to generate training data based on the selected label and the smell data.

(Supplementary Note 2)

The training data generation device according to Supplementary Note 1, in which

the information regarding the smell data is a speech regarding the smell data, and

the label candidate generation means generates the label candidates based on the speech.

(Supplementary Note 3)

The training data generation device according to Supplementary Note 1 or 2, in which

the information regarding the smell data is a text regarding the smell data, and

the label candidate generation means generates the label candidates based on the text.

(Supplementary Note 4)

The training data generation device according to any one of Supplementary Notes 1 to 3, in which

the information regarding the smell data is an image including a measurement target of the smell data, and

the label candidate generation means outputs the generation candidates based on the image.

(Supplementary Note 5)

The training data generation device according to any one of Supplementary Notes 1 to 4, in which

the information regarding the smell data is a trained model trained using a relationship between the smell data and the label, and

the label candidate generation means generates the label candidates based on the acquired smell data and the trained model.

(Supplementary Note 6)

The training data generation device according to Supplementary Note 5, in which

the trained model is trained using a relationship between the smell data and a sensory evaluation result for a smell.

(Supplementary Note 7)

The training data generation device according to Supplementary Note 5 or 6, in which

the trained model is trained using a relationship between the smell data and data indicating a chemical property of a measurement target of the smell data.

(Supplementary Note 8)

The training data generation device according to any one of Supplementary Notes 5 to 7, in which

the trained model is trained using a relationship between the smell data and data indicating a biological reaction when sniffing the smell.

(Supplementary Note 9)

A training data generation method including:

acquiring smell data and information regarding the smell data;

generating label candidates based on the information regarding the smell data;

outputting the generated label candidates;

receiving selection of a label from the output label candidates; and

generating training data based on the selected label and the smell data.

(Supplementary Note 10)

A learning model generation method including:

acquiring smell data and information regarding the smell data;

generating label candidates based on the information regarding the smell data;

outputting the generated label candidates;

receiving selection of a label from the output label candidates;

generating training data based on the selected label and the smell data; and

generating a learning model based on the generated training data.

(Supplementary Note 11)

A program recording medium that records a program for causing a computer to perform:

processing of acquiring smell data and information regarding the smell data;

processing of generating label candidates based on the information regarding the smell data;

processing of outputting the generated label candidates;

processing of receiving selection of a label from the output label candidates; and

processing of generating training data based on the selected label and the smell data.

### REFERENCE SIGNS LIST

10 sensor
11 terminal device
11a message requesting smell evaluation
11b message instructing selection of label to be registered
11c speech recognition result
11d label candidate
11e message instructing imaging of measurement target 13
11f message suggesting selection of partial region including
measurement target
11g extracted partial region
11h extracted partial region
11i message indicating selection of label
11j selection button “Yes”
11k selection button “No”
111 message requesting evaluation of smell of measurement target 13
12 evaluator
13 measurement target
20 time-series data
22 value indicating smell data
24 predicted value in label space
26 point corresponding to “tire”
100 training data generation system
1000 computer
1020 bus
1040 processor
1060 memory
1080 storage device
1100 input/output interface
1120 network interface
2000 training data generation device
2010 storage unit
2011 model storage unit
2020 acquisition unit
2030 label candidate generation unit
2040 output Unit
2050 receiving unit
2060 training data generation unit
2070 label candidate generation unit
2080 learning unit

What is claimed is:

1. A training data generation device comprising:
at least one memory storing instructions; and
at least one processor configured to access the at least one memory and execute the instructions to:
acquire smell data and information regarding the smell data;
generate label candidates based on the information regarding the smell data;
output the generated label candidates;
receive selection of a label from the output label candidates; and
generate training data based on the selected label and the smell data.

2. The training data generation device according to claim 1, wherein
the information regarding the smell data is a speech regarding the smell data, and
the at least one processor is further configured to execute the instructions to:
generate the label candidates based on the speech.

3. The training data generation device according to claim 1, wherein
the information regarding the smell data is a text regarding the smell data, and
the at least one processor is further configured to execute the instructions to:
generate the label candidates based on the text.

4. The training data generation device according to claim 1, wherein
the information regarding the smell data is an image including a measurement target of the smell data, and
the at least one processor is further configured to execute the instructions to:
output the generated label candidates based on the image.

5. The training data generation device according claim 1, wherein
the information regarding the smell data is a trained model trained using a relationship between the smell data and the label, and
the at least one processor is further configured to execute the instructions to:
generate the label candidates based on the acquired smell data and the trained model.

6. The training data generation device according to claim 5, wherein
the trained model is trained using a relationship between the smell data and a sensory evaluation result for a smell.

7. The training data generation device according to claim 5 or 6, claim 5, wherein
the trained model is trained using a relationship between the smell data and data indicating a chemical property of a measurement target of the smell data.

8. The training data generation device according to claim 5, wherein
the trained model is trained using a relationship between the smell data and data indicating a biological reaction when sniffing the smell.

9. A training data generation method comprising:
acquiring smell data and information regarding the smell data;
generating label candidates based on the information regarding the smell data;
outputting the generated label candidates;
receiving selection of a label from the output label candidates; and
generating training data based on the selected label and the smell data.

10. The training data generation method according to claim 9 further comprising:
generating a learning model based on the generated training data.

11. A non-transitory program recording medium that records a program for causing a computer to perform:
processing of acquiring smell data and information regarding the smell data;
processing of generating label candidates based on the information regarding the smell data;
processing of outputting the generated label candidates;
processing of receiving selection of a label from the output label candidates; and
processing of generating training data based on the selected label and the smell data.
